(19) United States
(12) Patent Application Publication
(10) Pub. No.: US 2002/0137093 A1
(43) Pub. Date: Sep. 26, 2002

(54) METHOD FOR CONTINUOUS MONITORING OF CHEMICAL SUBSTANCES IN FLUIDS

(76) Inventors: TEOFILO JOSE DIEZ-CABALLERO ARNAU, VALENCIA (ES); GUILLERMO RODRIGUEZ ALBALAT, VILLARREAL (ES); CRISTINA FERRER FERRER, BURRIANA (ES); ENRIQUE ESPINAS MARTI, MONCOFAR (ES); SERGIO MONTORO RODRIGUEZ, PATERNA (ES); VLADIMIR ERCHOV, VALENCIA (ES); ALEJANDRO MENDOZA PLAZA, VALENCIA (ES); TEOFILO DIEGO DIEZ-CABALLERO CARMONA, VALENCIA (ES)

Correspondence Address: RICHARD M GOLDBERG ESQ, 25 EAST SALEM STREET, SUITE 419, HACKENSACK, NJ, 07601

(21) Appl. No.: 09/449,852

(22) Filed: Nov. 26, 1999

(30) Foreign Application Priority Data
May 19, 1997 (ES) P9701073
May 7, 1998 (US) PCT/ES98/00127

(51) Int. Cl. G01N 33/53; C12Q 1/66

(52) U.S. Cl. 435/7.1; 435/8

(57) ABSTRACT

For the last two decades much work has been done in developing biosensors with specific applications (industrial, biomedical and environmental among others) that can now compete with the classical analytical technologies but these apparatus that are based on the retention of microorganisms in membranes, have intrinsic limitations. The two principal innovations brought forward in respect of the present technology are;

The continual availability of microorganisms when using a chemostat which allows the monitoring of samples with elevated toxicity, discarding the microorganisms used after every reading, eliminating the membrane which physically separates the medium to be analyzed and the transducer, thus allowing the amplification of the linear range of the signal and drastically reducing the time needed to reach a steady signal.

It is presented a microbiosensor system to monitor on-line chemical substances in fluids. Two examples of the application are presented: online determination of biochemical oxygen demand (BOD) in water and on-line determination of toxicity in water.

### TECHNOLOGICAL AREA OF THIS PATENT

Chemical and biochemical analysis. Environmental analysis.

System for FIA (Flow injection analysis) with discontinuous flow.

System for automatic analysis with autocalibration.

Application in many areas; environment, microbiological reactors, fermentors, biology, agricultural feeding and industries related to these given areas.

### STATE OF THE ART

During the last two decades there has been a growing development in the technology of biosensors as a result of the multidiscipline integration between the areas of enzymology, immunology and microbiology and those of sensors and signal transducers. This has allowed the development of biosensor with specific applications (industrial, biomedical or environmental to name a few) that can now compete with the classical analytical technologies. If enzymatic biosensors weoe to have a special application in the cases of repetitive identification of a molecule/substrate, for which the corresponding enzyme can be found, and if the use of immunobiosensors would allow, in the near future, the specific identification of any simple molecule or compound obtaining their respective antibodies, then microbiosensors could have many applications varying from environmental monitoring to the control of bioreactors

More recently, the advances in the field of genetic modification of microorganisms are now providing researchers with the elements necessary to design microbiosensors capable not only of detecting and quantifying specific compounds, but also of amplifying the generated signals in the detection, making use of the molecular biology of the modified microorganisms.

Among those configurations most studied until the present day for the design of microbiosensors you have to look at those of microbial membranes. in the is technology microorganisms, specific or not, are held in the interior of a membrane and are in contact with the signal transducers of the microbiosensor. The presence of substances that could affect the metabolism of the microbe produce a physical-chemical change in the membrane containing the microbes which is detected by the sensor and if the signal is suitably handled then the substances of interest can be measured. This technology has allowed the appearance on the market of commercial equipment with new and interesting specifications mainly in the environmental area (microbiosensors—BOD like those of Doctor Lange, Medingen or CKG among others)

Nevertheless these apparatus that are based on the retention of microorganisms in membranes have intrinsical limitations stemming from the idea and design.

To start with the microorganisms that are fixed to the membrane structure have little or no capacity to grow and are at risk in the presence of certain toxins or sterilising factors which can render the membrane unusable. Also the size and physical/chemical characteristics of the membrane can limit the diffusion of analytes across it and can reduce the concentration margin of the analytes for which the apparatus is capable of generating linear responses (E. Praet, V. Reuter, T. Gaillard, J.-L Vasel. “Bioreactors and biomenbranes for biochemical oxygen demand estimation” Trends in analytical Chemistry, vol. 14, no7, 1995).

Another big area of microbiosensors bases the functional design on the utilisation of a microbial reactor maintained in optimal conditions for microbial activity, into which is added the liquid sample, to be monitored, directly into the reactor, registering the signal produced by the transducer. In some of the variants of this type of microbiosensor you can adjust the volume. dilution or the flow of injection of the sample by means of automatic systems of feedback of the signal generated so that a predetermined value is maintained. Good examples of these microbiosensors are the respirometers like Rodtox (P. A. Vanrolleghem, et al. “An on-line Respirographic Biosensor for the Characterization of Load and Toxicity of Wastewater”, J. Chem. Tech. Biotechnol. 1994, 59, 321-333) and Stip. These types of microbiosensors are being used at the moment with specific applications (mainly BOD and toxicity) with satisfactory results but its design has functional limitations in many applications of interest. This is due to the overall change in the parameters of the microbiological reactor (concentration of microbes, functional change in the microorganisms, etc.) on introducing the sample to be analysed into the reactor, showing that on occasions there may be considerable concentrations of toxic chemical products capable of altering, over long periods of time, teh overall response of the microorganism sensors. Consequently i is difficult to maintain a base line of steady response making it impossible to analyse samples of high toxicity or, if you risk analysing these samples, reversibly or irreversibly damaging the microbiosensor.

The invention about to be described has changed the concept and design of microbiosensors making it possible to overcome a large number of limitations that they had before. Among the innovations that it has and the advantages it has over the actual technology of microbial membranes, in summary, are the following:

a) continual availability of microorganisms when using a chemostat with controlled growth parameters which allows the monitoring of samples of high toxicity discarding the microorganisms used in the microbial reaction unit after every measurement.

b) the microbial reactor unit or minireactor for the analysis is an independent compartment from the chemostat in which there is the continual cultivation of microorganisms, in the microbial reactor unit the samples for analysis are injected without interfering with the action of the continual cultivation of cells.

c) it eliminates the membrane which physically separates the medium to be analysed and the transducer, thus allowing the amplification of the range of the signal and reducing drastically the time necessary to reach a steady signal.

d) The introduction of the master solution as reference sample before and after the injection of the problem sample into the microbial reaction unit makes it possible to systematically autocalibrate the apparatus and obtain a stable base line of reference.

e) Discarding the contents of the microbial reaction unit that monitors the sample after every cycle makes it possible to inject into it a high ratio of: (volume of problem sample)/(total volume) which allows the detection of certain chemical compounds in small concentrations in the problem sample or with little capacity to generate a signal in the transducer.

It must be emphasized that the described invention does not refer to require a certain type of microorganism. The technology of the invention correspond to a microbiosensor that is made up of automatic devices, incubation unit, microbial reactor unit, and transducers, microprocessors PC, thermostat systems etc., which makes possible its use in several applications, utilizing specific microorganisms in each case, and not only those available at the time but those also that could be obtained in the future. In whichever of the possible applications of this patent, the advantages described in the previous paragraph will be obtained in whole or in part in comparison with the technology in use today.

### DESCRIPTION OF THE INVENTION

The technology and innovations arising from this patent, can be applied to a variety of types of microbiosensors, as much in the cultures used, pure or mixed, as the analysts monitored. specific molecules or unspecific substrates, or by the physicalchemical signal generated and the transducer used to quantify. As examples to explain the wide usage of the developed technology references will be made to: A/pure microbial strains, natural or genetically modified, that allow the monitoring of specific molecules using, for example optoelectronic signal transducers, and B/mixed microbial cultures capable of metabolizing or altering their metabolism when exposed to a wide variety of molecular species (analytes) present in the problem sample (example 1 for on-line BOD determination and example 2 for on-line Toxicity determination).

A/As a reference to this application is a short description of the use of genetically modified microorganisms into which have been inserted a determined genetic sequence via DNA recombinant technology, This sequence is made up of the genes called LUX which code the synthesis of the enzyme luciferase, which catalyses the oxidation reaction associated with the emission of one photon of light with a wavelength of 490 nm. This enzyme cornea from a procaryotic organism (Philip J. Hill, Stephen P. Denyer,” Rapid Assays based on in vivo luminiscence” Microbiology Europe, 16, May/June 1993), (Robert S. Burlage,” Living Biosensors for the Management and Manipulation of Microbial Consortia” Annual Review Microbiol., 48, 81-104).

Associated to the same promotor (region of the genome where the transcription of a gene occurs) are inserted the genes which code for the metabolic route of interest. When the specific metabolite to be measured (toxic) is present in the medium, the promotor of this genetic sequence is activated and transcribes both the genes that code for luciferase and those that code for the enzymes that catalyse the degradation of the said metabolite (Jorma Lampinen, Marko Virta, “Use of Controlled Lucifemase Expression To Monitor Chemicals Affecting Porotein Synthesis,” Applied and Environmental Microbioloy, August 1995, p. 2981-2989).

The analytes-molecules can be organic compounds that are considered toxic in the environment, like organochloro compounds. The bacteria metabolise these toxins, emitting light in the process, which can be detected and quantified, relating to the toxin present in the medium (S. Burlage,A. Palumbo,“Biolumuniscent Reporter Bacteria Detect Contaminants in Soil Samples” Applied Biochemistry and Biotechnology,vol. 45/46). The most innovative bacterial strains incorporate a constitutive control which consists of the insertion of a gene coding for the synthesis of a encaryotic luciferase into the bacterial chromosome, the expression of which is constitutive. And so you can detect the continual emission of light at 560 nm as an indicator of the good metabolic state of the biomass and in addition a light at 490 nm when a specific toxin is present in the medium (Angel Cebolla, F. Ruiz-Berraquero.” Stable Tagging of Rhizobium meliloti with the Firefly Luciferase Gene for Environmental Monitoring” Applied and Environmental Microbiology, August 1993, p. 2511-2519) (Wood Kv, Gruber M G.” Transduction in Microbial Biosensors Using Multiplexed Bioluminiscence.” Biosensors and Bioelectronics 11(3);207-214,1996), (Gu M B, Dhurjati P S,” A Miniature Bioreactor for Sensing Toxicity using recombinant bioluminiscent Escherichia coli Cells”, Biotechnology Progress,12(3);393-397, 1996); B/to go deeper in the function and technological aspect of this patent two detailed examples of the application of this patent to on-line Biochemical Oxygen Demand (BOD) determination and online Toxicity determination.

### EXAMPLE 1

A Working Example of the Application of the Invention in the On-line Monitoring of the Biochemical Oxygen Demand (BOD).

A microbiosensor system is presented for the online monitoring of chemical substances in fluids. The use of the system in the determination of the Biochemical Oxygen Demand will be described as a particular case and example of an application of the presented invention. The measurement of the Biochemical Oxygen Demand (BOD) is fundamental in controlling the function of the WwVIP (Waste Water Treatment Plant) and for the monitoring and observation of ecosystems. It is an analytical parameter that measures the oxygen used by microorganisms for the biochemical degradation of organic material contained in a sample during a specific incubation period at a given temperature. This information allows the adjustment of the oxygen needs of the biological reactor which is vital for the function of the WWTP.

The traditional ways of measuring the BOD require and incubation time of 5 days in the laboratory, which mean that the result obtained did not reflect the pollution of the water that needed to be treated at that moment in the WWTP.

As a consequence these analytical systems do not have much use in operative control processes that require sample quantification in shorter times. It is also an analysis with a considerable number of sources of variability in the results.

The BOD microbiosensor equipment, as an example of the application of the invention, is based on advanced and original technologies compared to other apparatus with the same purpose which are on the market today, and makes it possible to obtain a reading of BOD in liquid samples of diverse composition in only 15 minutes. It is then possible to:

a/ obtain up to date information about the performance of a WWTP or of the liquid volume analyzed.

b/ if necessary take instant correct measurements, being able to assess in a short time the effect produced by the applied measurements.

c/save energy used for aeration, one of the costly processes of the WWTP d centralize the monitoring of one or many WWTP by means of the remote capture of data.

e/ identify and analyze the presence of certain toxins in the liquid samples analyzed.

f/ determine the BOD even in liquid samples with elevated levels of toxins.

g/ autocalibrate every cycle, measuring the analytical response of the apparatus using internal reference samples.

h/ monitor the BOD of open systems in a wide variety of environmental temperatures.

In the following notes the function and components of the microbiosensor apparatus for the monitoring of chemical substances in fluids will be examined, in the specific case of the on-line determination of the BOD in water.

The configuration of the microbiosensor for the continual monitoring of chemical substances in fluids will now be described.

The microbiosensor is made up of an integrated system and includes

a/ units for the continual growth of microorganisms.

b/ microbal reaction units.

c/ transducer component (dissolved oxygen sensor in the examples of the BOD and Toxicity)

d/ a programmed hydraulic circuit for the handling of liquids.

e/ microprocessor for the control of the analytical process and the acquisition of the signal.

f/ PC device for the treatment of the data and communication with the PLC (microprocessor).

g/ integrated thermostabilization devices.

The microbiosensor should be able to use the apparatus. This systems is composed of peristaltic pumps to handle the samples to be analyzed, from the specified points in the WWTP into the apparatus and a sample preparation system that eliminates the solid suspensions that could interfere with the measurement of the BOD and toxicity.

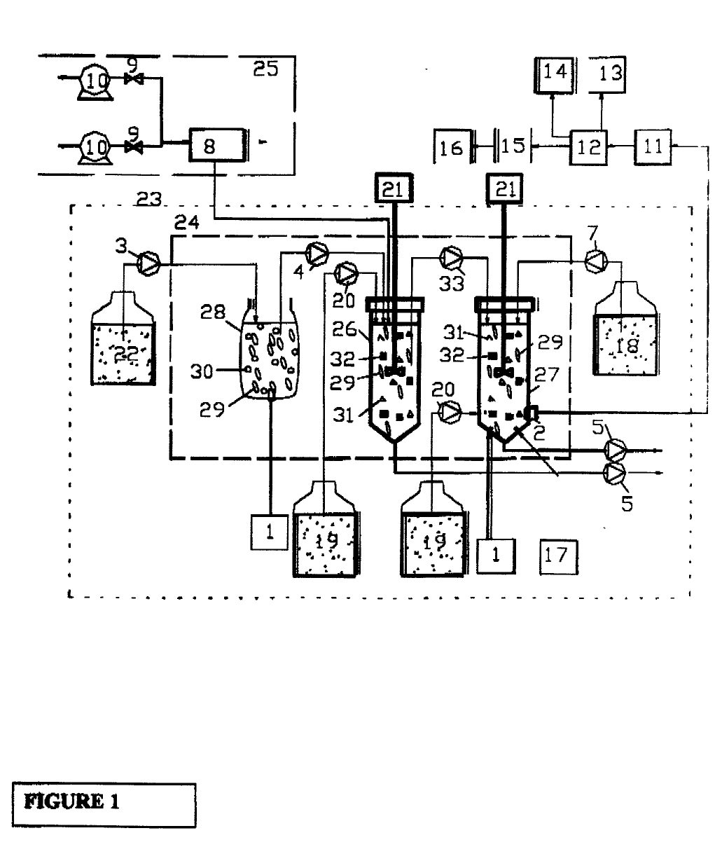

FIG. 1 shows the layout of the microbiosensor. The first stages of the functioning of the microbiosensor for the continual monitoring of chemical substances in fluid are laid out as follows: the system is based on the measurement of dissolved oxygen consumption that became when the used microorganisms metabolise the organic matter of the sample. The system uses the microorganisms (29) which are generated in a chemostat (28), in way that in each measurement cycle a predefined quantity of the microbial suspension is injected to the incubation unit (26), by a pristaltic pump (4).

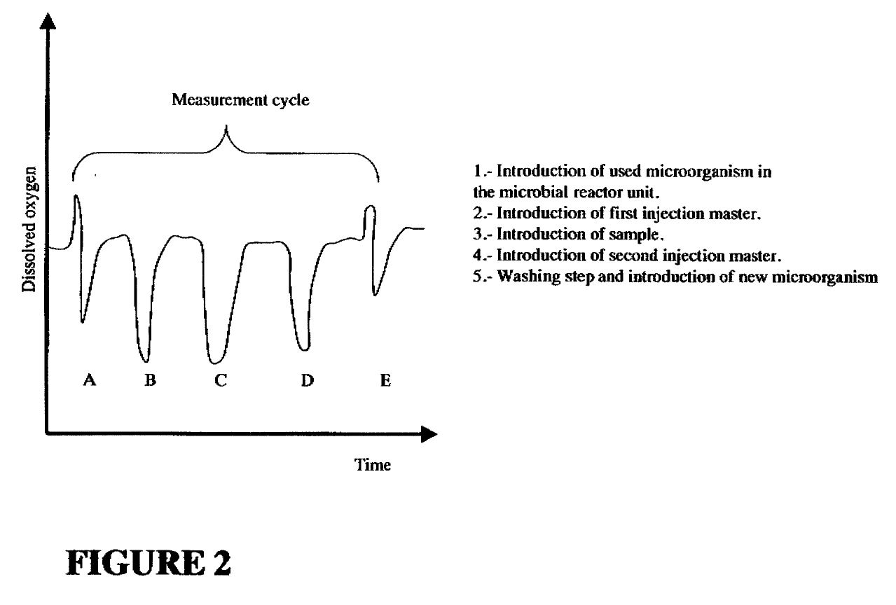

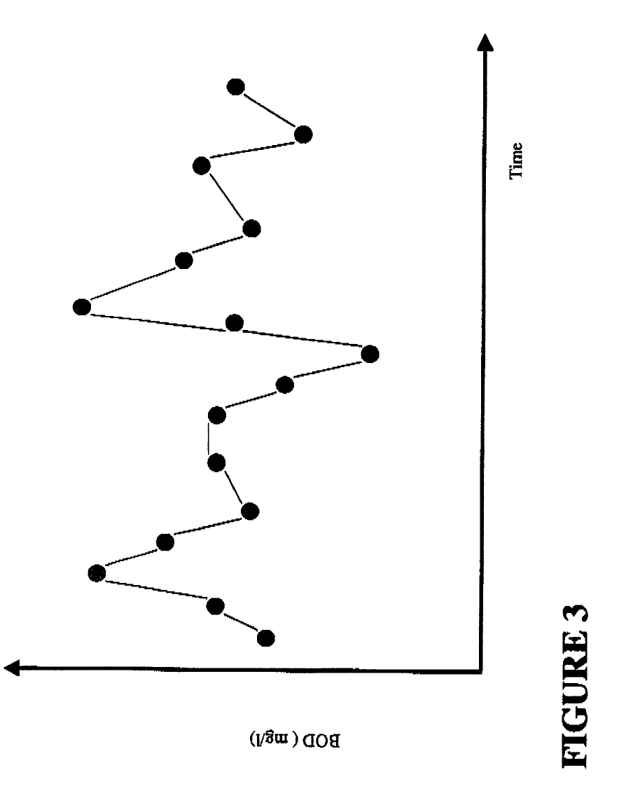

The chemostat (28) is continuously aerated by a aeration device (1′)contain structures of similar density to the liquid medium, hollow or with pores long enough to allow their colonisation by the used microorganisms are introduced into the interior of the chemostat and they serve as a concentrated microbial starter to accelerate the functional recovery of the continuous cultures. In cases of changes of apparatus or functional accidents whats more these structures favour the dissolving of oxygen in the suspension of microorganisms because they increase the time of contact between the air/liquid. The injected microbial suspension is homogenised by a stirrer (21). The homogenised suspension is passed to the microbial reactor unit (27), by a peristaltic pump (33). Both incubation unit (26) and microbial reactor unit (27) are whased by a washing solution which is injected by a peristaltic pump in each case (20 and 20′, respectively) from their corresponding reservoirs (19 and 19′, respectively). The chemostat (28) and the incubation unit (26) and the microbial reactor unit (27) are located in a thermostabilised compartment (24) to maintain a suitable temperature for the growth of used microorganisms (29). After the used microorganisms (29) had been extracted from chemostat (28), a predefined quantity of a nutrient solution, whose composition is specific from the used sensor microorganisms, is injected in the chemostat (28), by a peristaltic pump (3) from their reservoir (22) to maintain the volume of the chemostat (28) and to maintain the growth of the sensor microorganism, so in each measurement cycle, the chemostat (28) supplies equivalent quantities of the microorganisms in both composition, concentration and activity, Both washing solution (19 and 19′) and specific nutrient solution (22) are located in ai thermostabilised compartment (23) to maintain a low temperature. It make possible to optimise their conservation during the time period of equipment function. The microbial reactor unit is,; continuously aerated by means of an aeration device (1), so the microbial suspension passed from the incubation unit (26) to the microbial reactor unit (27) is continuously aerated. In such conditions, the concentration of dissolved oxygen (30) in the microbial suspension (29) within the microbial reactor unit (27), increases until an stationary state between the endogenous consumption of the dissolved oxygen by the used microorganisms (29) and the dissolved oxygen supplied by the aeration device (FIG. 2, A). In this situation a base line for the measurement has been reached. The concentration of dissolved oxygen (30) in the microbial reactor unit (27) is monitored during all the measurement cycle with the use of an dissolved oxygen electrode (2). In this case is a Clark type dissolved oxygen electrode, capable of measuring the oxygen concentration in the microbial reaction unit. The main job of the oxygen electrode is to determine the partial pressure of oxygen in liquids in accordance with the principles of Clark. The process of measurement is based on the separation of the sample and the electrode by a permeable membrane,

The reduction of oxygen at the working electrode is by the following reaction

O₂+2H₂O+4e.→4OH′

The oxidation of the reference electrode results from the consumption of electrons.

The steps between the fitting of the sensor and setting it working are shown here: the fixing in place and stretching of the membrane, the addition of the electrolyte, assembly of the hood over the sensor and the turning on of the fixed sensor.

When the base line for the measurement is reached, a predefined quantity of a master is injected in the microbial reactor unit (27) containing the suspension of the used microorganisms (29), by a peristaltic pump (7) from their reservoir (18). The injected master is mixed with the suspension of the used microorganisms (29). The master have a known valour of the Biochemical Oxygen Demand. The reservoir of the master solution (18) is located in a thermostabilised compartment (23) to maintain a low temperature. When the master has been injected in a predefined quantity and has been mixed with the aerated microbial suspension (29), the used microorganisms consume the exogenous organic matter, which has been added with the master. This metabolic process require a dissolved oxygen consumption (30) by the used microorganisms (29), so the concentration of dissolved oxygen in the microbial reactor unit (27) decreases (FIG. 2, B). When all of the exogenous organic master of the master has been metabolised by the used microorganisms (29) the dissolved oxygen concentration (30) increases within the microbial reactor unit (27) reaching other one the stable base line for the measurement.

The biochemical process of the dissolved oxygen consumption by the microorganisms when they metabolise organic matter is named respiration. So, the analytical signal obtained by the microbial respiration of the master is named respirometric peak of the master

Following this, a external circuit (25) which comprises a combination of pumps (10) and electrovalves (9) and a sample preparation unit (8) takes during enough time the water running that will be analysed ensuring that the analysed sample will be fresh sample. After a predefined time of running water within the external circuit (25), a peristaltic pump (6) inject a predefined quantity of sample in the thermostabilised incubation unit (26), where the sample is at tempered and homogenised by means of a stirrer (21).

This sample is passed from the incubation unit (26) to the microbial reactor unit (27). When the sample has been injected in a predefined quantity and has been mixed with the aerated microbial suspension (29), the used microorganisms consume the exogenous organic matter, which has been added with the sample. This metabolic process require a dissolved oxygen consumption (30) by the used microorganisms (29), so the concentration of dissolved oxygen in the microbial reactor unit (27) decreases (FIG. 2, C). When all of the exogenous organic matter of the sample has been metabolised by the used microorganisms (29) the dissolved oxygen concentration (30) increases within the microbial reactor unit (27) Teaching other one the stable base line for the measurement.

The biochemical process of the dissolved oxygen consumption by the microorganisms when they metabolise organic matter is named respiration. So, the analytical signal obtained by the microbial respiration of the sample is named respirometric peak of the sample.

The comparison between the respirometric signal of the master and the respirometric signal of the sample, allows the determination of the Biochemical Oxygen Demand of the sample.

When the base line for the measurement is other one reached, a predefined quantity of a master is injected other one in the microbial reactor unit (27) containing the suspension of the used microorganisms (29), by a peristaltic pump (7) from their reservoir (18). The injected master is mixed with the suspension of the used microorganisms (29). The master have a known valour of the Biochemical Oxygen Demand. The reservoir of the master solution (18) is located in a thermostabilised compartment (23) to maintain a low temperature. When the master has been injected in a predefined quantity and has been mixed with the aerated microbial suspension (29), the used microorganisms consume the exogenous organic matter, which has been added with the master. This metabolic process require a dissolved oxygen consumption (30) by the used microorganisms (29), so the concentration of dissolved oxygen in the microbial reactor unit (27) decreases (FIG. 2, D). When all of the exogenous organic matter of the master has been metabolised by the used microorganisms (29) the dissolved oxygen concentration (30) increases within the microbial reactor unit (27) reaching other one the stable base line for the measurement.

The biochemical process of the dissolved oxygen consumption by the microorganisms when they metabolise organic matter is named respiration. So, the analytical signal obtained by the microbial respiration of the master is named respirometric peak of the master (second master injection).

The comparison of both respirometric signals of the first and second master injections, before and after the sample injection, respectively, allows the determination of toxicity of the sample because the sample toxicity have an effect on the consumption of the dissolved oxygen (30) by the used microorganisms (29) if this sample contain one or more toxic compounds (32). The rate to consume dissolved oxygen for the first master (before the sample injection) is smaller than the rate to consume dissolved oxygen in the second master (after the sample injection).

One of the measurement cycle has been concluded, the mixture contained in the microbial reactor unit (27) is empty out to the waste by means of a peristaltic pump (5). Following this, a predefined quantity of a washing solution is injected from their reservoir (19 and 19′) to the microbial reactor unit (27) and the incubation unit (26), by corresponding peristaltic pumps (20 and 20). After a predefined period of time, the content is empty out by the corresponding peristaltic pumps (5 and 5′) to the waste. The system is prepared to initialise other analytical other measurement cycle (FIG. 2, D).

The control system of the analytical process consists of a computer (PC) (12) that continually supervises the operation of a programmed microprocessor (PLC) (11), which carries out the instructions of the specific analysis protocol controlling between other parameters the following: volumes and sequences of the taking of the components of the process, reaction times before the reading of results, obtaining the signal generated by the transducer, an autocheck of the system and finally the calculation and presentation of results.

The computer (PC) carries out the complex processing of the signals from the transducers and permits the modification of the controls carried out by the programmable microprocessor depending on the needs of the measurements or the conditions of the function of the system. The incorporation modems into the computer system allows the microbiosensor equipment to be remote controlled by means of a telephone connect (16), thus making it possible to centralise the control of various microbiosensor situated at different WWTP. In addition, the apparatus has a monitor (13) and a printer (14).

As an example, the control software and acquisition of data from the microbiosensor of the present invention, in the particular case of the BOD, will be described.

The BOD microbiosensor is controlled by specialised software. The apparatus of the microbiosensor includes a PC computer (12) and a microprocessor (11). The control programmer implement the following functions:

the graphical design of a diagram showing the function of the devices with respect to time for a specific protocol.

control of the function of the electric devices of the fluid circuit.

acquisition of the signals from the sensor(s).

display of the obtained data on the screen of the computer.

preliminary treatment of the data.

storage of the data.

The software of the microbiosensor includes two main programmes and various additional programmes (for the remote control of the apparatus, to construct graphs, and so on). The programme of the PLC microprocessor (11) allows for the control of the function of the apparatus independent of the PC computer (12), whilst the programme of the PC allows for the receiving, storing and analysis of the data.

The programme of the PC computer (12) receives every 30 seconds the data from the PLC microprocessor (11) and stores them in a file on a hard disk. At the same time, the programme displays these data in graphical form and analyses the changes observed during the cycle. This graph of dissolved oxygen is shown in a window where the operator can note any point of interest and calculate the areas under the peaks that correspond to the respirometric signals of the microbiosensor to the sample.

The PC programme prepares two screens more: one called diagram of time and the other one diagram of the function of the apparatus. The diagram of time allows you to observe the function of the cycle using the graphs of activation and inactivation of the devices in the fluid circuit. The function diagram is a window that helps to the better understanding of the function of the microbiosensor and allows the control of the electrical devices of the apparatus (the peristaltic pumps and the electrovalves). The hardware of the PLC microprocessor (11) is designed so that the exchange of messages between the microprocessor and the PC computer (12) can occur without interrupting the programme of the microprocessor.

The function diagram represents the structure of the fluid circuit of the microbiosensor This diagram permits the control of the peristaltic pumps and electrovalves from the screen of the PC computer. Any pump or electrovalve can be activated simply by moving the cursor onto its image and pushing the left key of the mouse. The image of the pump changes. If you press again the image of the activated pump it stops. In the same way the electrovalve can be activated inactivated.

The diagram of dissolved oxygen shows the up to date data obtained during the cycle. The data represents the 600 last dissolved oxygen measurements (taken as often as required). Normally the peaks of sample measurements and the master peaks appear on the diagram. The area of the peaks represent the biochemical oxygen demand (BOD). When a new peak appears, the new area is calculated automatically and the result of the calculation is shown in the bottom part of the window in the information bar. It is also possible to calculate the area of any peak on the graph by using the “Area” command in the menu. By using the “Start Recording”, “Finish Recording” and “Base line” commands it is possible to mark the limits for the integration of the respirometric graph. By using the “Calculate” command the marked area will be calculated and the result will be shown on the screen. The diagram of time shows where in the analytical cycle the apparatus is. The state of the peristaltic pumps is seen as an activated unit (the upper signal) if the pump is activated, or at zero if the pump is switched off.

The apparatus can also be controlled by using the menus “Windows”, “Commands”, “User”. With the menu “Windows” the operator chooses one of the three windows to control the apparatus (Dissolved Oxygen, Diagram of Time or Boxes Diagram). By pressing one of the items in the “windows” menu, the corresponding window will be shown in front of the other and the operator can then use the diagram. The “User” menu serves to regulate the level of access to the commands (for example, a supervisor can change the properties of the cycle but another operator will not have access to this command). The “Command” menu serves to activate mini cycles in the fluid circuit.

The command “Stop the Cycle” is carried out at a programmed time and includes the activation of various pumps (those of washing and emptying).

The command “Change Cycle Parameters” serves to change the function times of the cycle.

The command “Microorganisms” activates the microorganism pump and after that, the pump for the feeding the mother reactor.

The command “Master” activates the master solution pump after a predetermined time.

The command “Inlet Sample” starts the part of the cycle involved in the taking of it sample from the entrance to the WWTP. At first, the external pump for the sample is activated at the entrance to the WWTP allowing the sample to reach the apparatus. After a time, both the internal peristaltic pump and the electrovalve for the injection of the sample into the microbial reaction unit are activated.

The command “Outlet Sample” starts the part of the cycle involved in the taking of a sample from the outlet of the WWTP which follows the same pattern as the command described above, for the taking of a sample from the inlet of the WWTP.

The FIG. 3 shows the graphic of the calculated BOD versus time.

### EXAMPLE 2

A Working Example of the Application of the Invention in the On-line Monitoring of the Toxicity.

By means of an external circuit (25) which comprehends a combination of pumps (10) and electropumps (9) and a unit of sample preparation (8), the water stream is apirated and circulated during the sufficient time to assure that the analysed sample would be fresh. After a determined time of circulation of the liquid stream by the external circuit (25), a peristaltic pump (6) injects a pre-determined quantity of sample to analyse in the incubation unit (26) thermostabilised, where the sample reach a determined temperature and is homogenised by means of a stirrer device (21).

Afterwards, in the incubation unit (26) is added, from the chemiostat (28), a pre-determined quantity of suspended microorganisms (29), by means of a peristaltic pump (4). The suspended microorganisms (29), sent from the chemiostat (28) to the incubation unit (26), is mixed here with the sample (31) by means of a stirrer device (21). The half part of this homogeneous mixed is sent immediately to the microbial reactor unit (27) by means of a peristaltic pump (33). The microbial reactor unit (27) contains a dissolved oxygen electrode (2) which permits to measure the concentration of dissolved oxygen in the microbial reactor unit (27) in the whole measurement cycle.

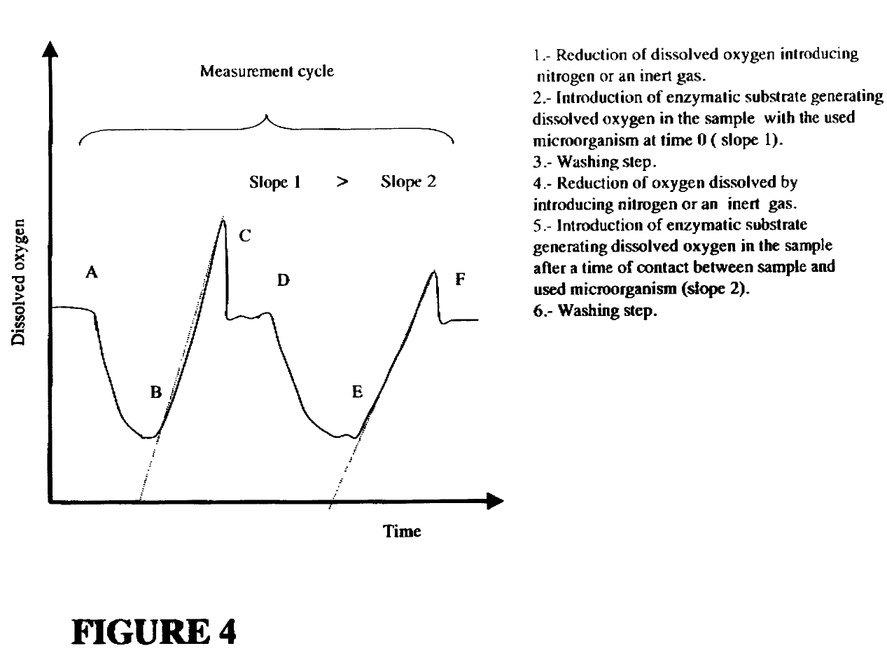

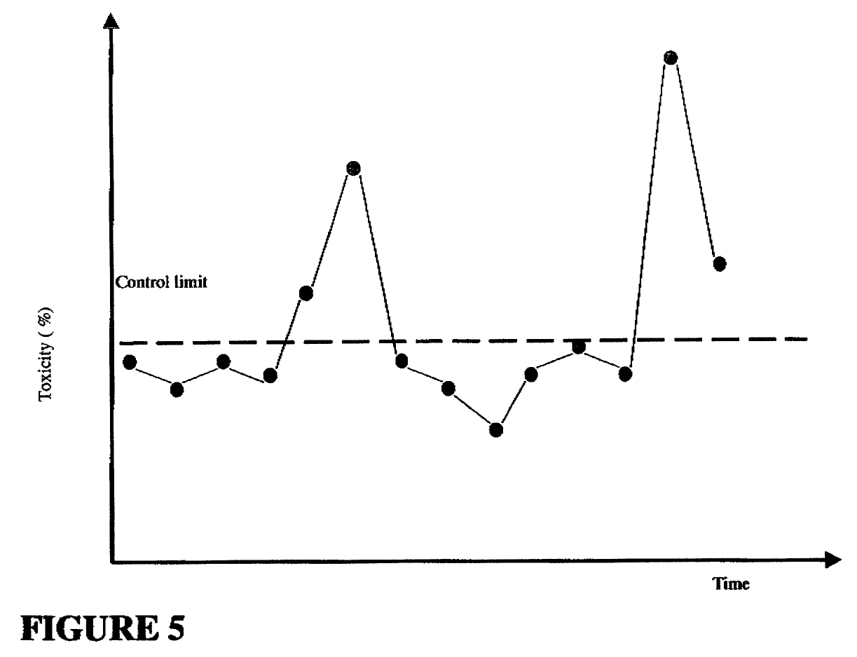

Afterwards, in the mixture contained in the microbial reactor unit is injected (27) gaseous nitrogen or inert gas from its reservoir (17), which permits to reduce the dissolved oxygen concentration in the mixture until it reaches a predetermined value (FIG. 4, A). Then a specific enzymatic substrate of the used microorganisms is added to the deoxygenated mixture (29) by means of a peristaltic pump (7) from its reservoir (18) to the microbial reactor unit (27). The microorganisms (29) decompose this substrate by means of an enzymatic reaction producing oxygen as one of the outputs (30). Therefore, as a consequence of the microbial decomposition of the enzymatic substrate is produced an increment of the dissolved oxygen concentration in the reaction mixture contained in the microbial reactor unit (27). The increment of the dissolved oxygen concentration in the reaction mixture is measured in a predetermined time, that is, the speed of dissolved oxygen production (FIG. 4, B). This value is proportional to the number of microorganisms contained in the sample.

Afterwards, the microbial reaction unit (27) is voided by means of a peristaltic pump (5). After concluding the voidance of the content of the microbial reaction unit (27) is injected a predetermined quantity of a washing solution from its reservoir (19) by means of a peristaltic pump (20). After a sufficient time for the washing of the microbial reactor unit (27), it is voided the content with a peristaltic pump (5)(FIG. 4, C).

The other half part of the mixture formed by the microorganisms (29) and the sample to analyse from the incubation unit (26), is injected in the microbial reactor unit (27) by means of a peristaltic pump (33), after a predetermined time of contact in the incubation unit (26).

Afterwards, in the mixture contained in the microbial reactor unit (27) is injected gaseous nitrogen or inert gas from its reservoir (17), which permits to reduce the concentration of dissolved oxygen in the mixture until it reaches a predetermined value (FIG. 4, D). Then an specific enzymatic substrate of the used microorganisms is added to the deoxygenated mixture (29) by means of a peristaltic pump (7) from its reservoir (18) to the microbial reactor unit (27). The microorganisms (29) decompose this substrate by means of an enzymatic reaction producing oxygen as one of the outputs (30). Therefore, as a consequence of the microbial decomposition of the enzymatic substrate is produced an increment of the dissolved oxygen concentration in the reaction mixture contained in the microbial reactor unit (27). The increment of the dissolved oxygen concentration in the reaction mixture is measured in a predetermined time, that is, the speed of dissolved oxygen production (FIG. 4, B). This value is proportional to the number of microorganisms contained in the sample. Finally, the microbial reactor unit and incubation unit are voided and washed in the same form that before (FIG. 4, F). If the analysed sample (31), which is in contact with the microorganisms (29), contains a toxic compound or a mixture of toxic compounds (32), this will affect the microorganisms activity (29) during the predetermined time of contact in the incubation unit (26). Therefore, the increment of dissolved oxygen produced by the decomposition of the enzymatic substrate in a determined time will be less, that is, the speed of production of dissolved oxygen will be less as a consequence of the toxic effect of the sample. The comparison of the speeds of oxygen production by two aliquots in which the sample analysed (31) has been divided, the first for a zero contact time with the microorganisms (29) in the incubation unit (26) and the second after a predetermined contact time with the microorganismns (29) in the incubation unit (26), permits to calculate the toxicity of the analysed (31).

### Functional Diagram

1. Aeration device for microbial reactor unit

1′ Aeration device for chemostat

2. Dissolved oxygen electrode

3. Supply pump

4. Microorganism injection pump

5. Emptier pump

6. Sample injection pump

7. Master injection pump

8. Sample preparation pump

9. Electric valves

10. Water sample pumps

11. Controller

12. Computer

13. Monitor

14. Printer

15. Modem

16. Telecontrol device

17. Nitrogen

18. Master (BOD) or Enzymatic Substrate (TOX)

19. Washing solution for microbial reactor unit

19′ Washing solution for incubation unit

20. Washing pump for microbical reactor unit

20′ Washing pump fro incubation unit

21. Stirrer

22. Supply solution

23. Cooled thermostabilised unit

24. Warmed thermostabilised unit

25. External circuit to the optional equipment for waste water treatment stations

26. Incubation unit

27. Microbial reactor

28. Chemostat

29. Microoorganisms

30. Dissolved oxygen

31. Sample

32. Toxin

33. Incubated mixture injection pump

1.- A method for continous monitoring of chemical substances in fluids characterised in that the microorganisms used are obtained by means of constant growth in an automatic chemostat.

2.- A method for continous monitoring of chemical substances in fluids, in accordance with claim 1, characterised by the use of a microbial reaction unit with controlled constants into which, following the analytical protocol, are transferred automatically aliquots both used sensor microoganisms from the chemostat and of master samples, problem samples to be analysed and liquid mediums necessary to optimise the analytical protocol, and whose content is discarded at the end of each analytical cycle.

3.- A method for continous monitoring of chemical substances in fluids, in accordance with claims 1 and 2, characterised by the use of microorganisms whose metabolism is modified in proportion to the concentration of chemical substances of interest present in the liquid sample to be analysed.

4.- A method for continous monitoring of chemical substances in fluids, in accordance with claims 1 to 3, characterised by the use in the microbial reaction unit or in the liquid circuits of specific sensors to measure the physical/chemical signals generated by the microbial metabolism produced by chemical substances of interest present in the sample.

5.- A method for continous monitoring of chemical substances in fluids, in accordance with claims 1 to 4, characterised by the determination of the Biochemical Oxygen Demand (BOD), by means of a dissolved oxygen electrode situated in the mictobial reaction unit.

6.- A method for continous monitoring of chemical substances in fluids, in accordance with claims 1 to 5, characterised by the use of a luminometer to monitor the electromagnetic radiation produced by the luciferase enzyme of microorganism used, in response to the specific chemical substances present in the analysed sample.

7.- A method for continous monitoring of chemical substances in fluids, in accordance with claims 1 to 6, characterised by the use of an automatic cleaning system between sucessible analytical cycles by means of a jet of washing liquid which means that the sensor, the microbial reaction unit and the liquid circuits are kept in optimal conditions.

8.- A method for continous monitoring of chemical substances in fluids, in accordance with claims 1 to 7, characterised by the use in the microbial reaction unit of sensors for the determination of the analytical parametets in the cycle, like pH, conductivity, etc. complementary to the specific sensor of the physical/chemical signals produced by the presence of the chemical substances of interest.

9.- A method for continous monitoring of chemical substances in fluids, in accordance with claims 1 to 8, characterised by the use of integrated software and by the treatment of the signals generated by the specific sensors of the apparatus that allows the continuous monitoring of the corresponding parameters.

10.- A method for continous monitoring of chemical substances in fluids, in accordance with claims 1 to 9, characterised by the use of software and of a microprocessor to automatically control the integrated elements of the apparatus and which allow the selection of the corresponding parameters for the specific analytical protocol via the screens showing the diagram of time of the process, box diagrams of the apparatus and screens with the graphs of the registered signals.

11.- A method for continous monitoring of chemical substances in fluids, in accordance with claims 1 to 11, characterised by the use of software and modem for the transmission, via telephone or radio, of the analysed data and for the remote control of the parameters and analytical protocol of the apparatus.

12.- A method for continous monitoring of chemical substances in fluids, in accordance with claims 1 to 11, characterised by the use of software adjustable to specific analytical protocol that the user designs for the specific applications.

13.- A method for continous monitoring of chemical substances in fluids, in accordance with claims 1 to 12, characterised by the use of devices for the independent thermo stabilisation which allows the maintenance of preset temperatures in the compartments of the system that require it, making possible the function of the apparatus even in conditions of extreme surrounding temperatures.

14.- A method for continous monitoring of chemical substances in fluids, in accordance with claims 1 to 13, characterised by the alimentation of the microorganisms whit a specific liquid culture medium for each type of microorganism used and automatic and programmed feeding medium distribution system which allow for continual growth and metabolic activity of the microorganisms used.

15.- A method for continous monitoring of chemical substances in fluids, in accordance whith claims 1 to 14, characterised by a sample preparation system, placed in the circuit used to take the problem samples, which allow you to obtain suitable samples free of solids components in suspension.

16.- A method for continous monitoring of chemical substances in fluids, in accordance with claims 1 to 15, characterised by the use of a sterilisation system using an ultraviolet light that prevents the growth of microbes in the liquid feeding medium of the microorganisms and in the washing liquids.

17.- A method for continous monitoring of chemical substances in fluids, in accordance with claims 1 to 16, characterised by the automatic mechanism of injection and analysis in each cycle, of pre-set volumes of reference sample, of specific composition, that allow the autocalibration of the system, the detection of toxic effects and the detailed study of the analysed response.

18.- A method for continous monitoring of chemical substances in fluids, in accordance with claims 1 to 17, characterised by the use of an automatic system for the continual growth of the microorganisms, contained in their corresponding chemostats that allows for the taking of aliquots of the microorganisms with stable concentrations and metabolic activities, for the fresh use in every analytical cycle.

19.- A method for continous monitoring of chemical substances in fluids, in accordance with claims 1 to 18, characterised by the use of automatic mechanisms that allow the metabolic reactivation and the taking of aliquots of the selected microorganisms with reproducible and predetermined specifications, seeding the reactor with lyophilised or cold preserved microorganisms.

20.- A method for continous monitoring of chemical substances in fluids, in accordance with claims 1 to 19, characterised by the solid elements in suspension in the microorganisms continual growth reactor, whose structure with holes and pores crate an microenvironment for the colonisation of the microorganisms.

21.- A method for continous monitoring of chemical substances in fluids, in accordance with claims 1 to 20, characterised by the use of macro and mini reactors preventing in microbial colonisation on the internal surfaces by means of the movement of the solid structures in suspension caused by forced liquid movement.

22.- A method for continous monitoring of chemical substances in fluids, according to claims 2 to 21, characterised in that it allows, by means the microprocessor, to maintain both predefined quantities and times a mixture between the sample which toxicity is analysed and the used microorganism.

23.- A method for continous monitoring of chemical substances in fluids, according to claims 2 to 22, characterised in that it comprises an automatic system for introduction of nitrogen gas or an inert gas in microbial reaction unit, in order to reduce the oxygen dissolved concentration in the mixture between the sample which toxicity is analysed and the used microorganism.

24.- A method for continous monitoring of chemical substances in fluids, according to claims 2 to 23, characterised in that a specific enzymatic substrate of the used microorganism, hydrogen peroxide, is introduced in the microbial reaction unit, generating dissolved oxygen in a quantity that is proportional to metabolic activity of the used microorganism, modified by the sample which toxicity is analysed.
